(19) United States
(12) Patent Application Publication
(10) Pub. No.: US 2010/0143974 A1
(43) Pub. Date: Jun. 10, 2010

(54) Process for Sugar Production from Lignocellulosic Biomass Using Alkali Pretreatment

(76) Inventors: Chang-Ho Chung, Kyungki-do (KR); Donal F. Day, Baton Rouge, LA (US)

Correspondence Address: PATENT DEPARTMENT;TAYLOR, PORTER, BROOKS & PHILLIPS, L.L.P, P.O. BOX 2471, BATON ROUGE, LA, 70821-2471, US

(21) Appl. No.: 12/525,354

(22) PCT Filed: Jan. 31, 2008
(86) PCT No.: PCT/US08/52657

§ 371 Date: Jan. 15, 2010

Related U.S. Application Data
(60) Provisional application No. 60/887,684, filed on Feb. 1, 2007.

(51) Int. Cl. C12P 7/10; C12P 19/00

(52) U.S. Cl. 435/72; 435/165

(57) ABSTRACT

We have discovered a new method to treat biomass with alkali, for example lime. The lime and lignin was sufficiently removed from the treated biomass b> squeezing with a high pressure device to remove alkali and other potential inhibitors of the cellulase enzymes added for sacchaπfication. The resulting fibrous material was rapidly solubilzed by cellulases, even at solid loads ranging from 10 to 30% (w/w) without inhibitory effects on the cellulase activity. The lime pretreatment removed lignin effectively and left the cellulose and hemicellulose almost intact. The method yielded a biomass with structure capable of being enzyme solubilzed and fermented readily at a solids loading of 10-30% for a production of ethanol.

The benefit of the filing date of provisional application Ser. No. 60/887,684, filed 1 Feb. 2007, is claimed under 35 U.S.C. §119(e) in the United States, and is claimed under applicable treaties and conventions in all countries.

The development of this invention was partially funded by the United States Government under grant number DE-FG36-04G014236 from the United States Department of Energy. The United States Government has certain rights in this invention.

### TECHNICAL FIELD

The invention relates to a method for an alkali pretreatment for lignocellulosic biomass to be used in the process of producing simple sugars for fermentation, potentially to ethanol, and other useful by-products.

### BACKGROUND ART

The daily consumption of gasoline in the United States was estimated to be about 400 million gallons in 2004. The recent energy policy set a goal to replace 30% of the 2004 level of consumed gasoline by ethanol by the year 2030. In 2004, the amount of ethanol used for transportation was only about 2%. Most of the ethanol in the U.S. is produced from corn grain or from sugars from sugarcane and sugar beet. Interestingly, if the total amount of corn grain produced in 2005 in the U.S. were used for ethanol production for transportation fuel, only 12% of transportation gasoline is estimated to be replaced. (J. Hill et al., Proc. Natl. Acad. Sci. USA, vol. 103(30), pp. 11206-10; Epub 2006 Jul. 12 (2006)). Currently, the primary use of corn meal is for animal feed, followed next for the food industry, and then for ethanol production. Thus, an alternative biomass source that does not compete with food uses is required to meet the goal of the 2005 government policy. Additional biomass sources include agricultural residues or wood, including switchgrass, waste paper, corn grain, corn cobs, corn husks, corn stover, wheat, wheat straw, hay, barley, barley straw, rice straw, sugar cane bagasse, other grasses, sorghum, soy components, trees, branches roots, leaves, wood chips, sawdust, shrubs, bush, and combinations thereof.

The cost to produce bioethanol from lignocellulosic biomass is higher than from corn because of the expense of collection, pretreatment and enzymes. The cost of enzymes eventually may be significantly reduced based on improved production processes and use of genetically modified strains. Current pretreatment methods for biomass include use of acid or alkali, high temperatures (ranging from 50° C. to 220° C.), pressure explosions or combinations thereof, and additions of various other chemicals. Examples of chemicals commonly in use include sulfuric acid, hydrogen peroxide, ammonia and lime. Among these different treatments, dilute acid is considered as having the highest potential as a pretreatment for cellulosic ethanol production because of its relatively low cost. Low concentrations of sulfuric acid (0.005 to 0.07 g of sulfuric acid/g of dry solid biomass) with temperatures above 160° C. have been used to break the structure of lignocellulosic biomass and increase the hydrolyzation of cellulose. However, this technique produces inhibitors for the enzyme initiated hydrolysis (e.g., cellulases and hemicellulases), as well as fermentation inhibitors. A detoxifying step, such as overliming, is required prior to enzyme hydrolysis. Even though overliming is an effective method for reducing of the toxicity of inhibitors from acid pretreatment, the highly alkaline pH required (9 to 11) results in sugar loss and requires pH reduction prior to enzyme hydrolysis (Mohagheghi, Ali, Ruth, Mark, and Schell, Daniel J. 2006. Conditioning hemicellulose hydrolysates for fermentation: Effects of overliming pH on sugar and ethanol yields. Process Biochemistry, 41:1806-1811)

Alkaline treatments have been used in paper pulping for years. Sodium hydroxide effectively removes lignin from biomass, leaving the cellulose and hemicellulose for enzyme hydrolysis. However. NaOH is too expensive for use in the quantities required for a pretreatment method for the biomass amount required for bioethanol. Lime (calcium oxide (CaO) or calcium hydroxide (Ca(OH)₂)) is the least costly alkaline chemical, and is used in numerous industries from sugar to steel production. Lime is more environmentally friendly than other potential basic chemicals since it can be easily recovered as the calcium salt. (See U.S. Pat. No. 5,693,296) For example, carbon dioxide (CO₂) from fermentation and/or flue gas from a furnace can be used to recover the calcium (Ca) as calcium carbonate or bicarbonate. Consequently these chemicals can be used to regenerate calcium oxide by heating in a kiln. (Karr, William E., and Holtzapple, Mark T., 2000. Using lime pretreatment to facilitate the enzyme hydrolysis of corn stover. Biomass and Bioenergy, 18: 189-199, Chang et al., “Lime pretreatment of crop residues bagasse and wheat straw,” Applied Biochemistry and Biotechnology, vol. 74, pp. 135-159 (1998)). Lime pretreated biomass has been shown to be easily hydrolyzed by enzymes. Acetic acid was used to lower the pH of lime-pretreated biomass, from about pH 11-12 to about pH 4.8, which is the optimal pH for the cellulase enzymes. An inhibitory effect on the cellulase was found due to the calcium acetate that was formed as the salt concentration increased. (See U.S. Pat. Nos. 5,693,296 and 5,865,898; and Karr, William E., and Holtzapple, Mark T., 2000. Using lime pretreatment to facilitate the enzyme hydrolysis of corn stover. Biomass and Bioenergy, 18: 189-199). Lime was investigated as a pretreatment for biomass, and shown to be effective across a range of temperatures, treatment times, and different loadings. The addition of an oxidizing agent such as oxygen or an oxygen-containing gas during lime pretreatment was recommended to increase the removal of lignin. Lime pretreatment has also been used for bagasse, but cellulases were found not to achieve solubilization of biomass higher than 5% (w/v) loading. (Chang et al., “Lime pretreatment of crop residues bagasse and wheat straw,” Applied Biochemistry and Biotechnology, vol. 74, pp. 135-159 (1998)). In addition, soluble lignin and hemicellulose sugars such as xylose and arabinose after pretreatment produced cellulase inhibitors such as furfurals and furaldehydes. For better enzyme hydrolysis to simple sugar production, these compounds must first be removed.

Lignin components, mainly p-coumaric acid and ferulic acid, are found in biomass as esterified to cell wall polysaccharides. (Higuchi, T., Ito, Y., Shimada, M., and Kawamura, I., (1967) Phytochemistry 6, 1551). Alkali, e.g., Ca(OH)₂ or NaOH, reacts with these phenolic acids, even at room temperature, breaking the ester bonds from cell wall polysaccharides and forming salts. This addition of alkali (saponification) has been also shown to remove acetyl groups from acetic acid pulp resulting in improvements in cellulose hydrolysis (Pan, Xuejun, Gilkes, Neil and Saddler, Jack. N. 2006. Effect of acetyl groups on enzymatic hydrolysis of cellulosic substrates. Holzforschung, 60:398-401). Treatment with alkaline chemicals is known to improve the cellulose digestibility of non-woody plants as well. (Gould, J. Alkaline peroxide treatment of nonwoody lignocellolosics. U.S. Pat. No. 4,649,113). Chang et al. (1998) reported no removal of ash, xylan or glucan and 14% lignin was removed from washed bagasse after treatment with 0.1 g lime (as Ca(OH)2)/g of dry biomass at 120° C. for 1 hr.

To make bioethanol from biomass competitive to corn-based bioethanol, a solid loading higher than 20% (w/w) is required. High-solids loadings lower energy requirements and enhance ethanol recovery. The limitations of current biomass pretreatment technologies are: (1) few pretreatment methods work at levels greater than 10% solids; (2) cost of most pretreatment chemicals is high; and (3) most methods require significant particle size reduction, a grinding step, an energy intensive process, prior to pretreatment.

### DISCLOSURE OF INVENTION

We have discovered a new method to use alkali for biomass pretreatment. This new method included the following steps: (1) Raw biomass with sizes up to 10 inches in length (for example, sugarcane bagasse) was mixed with lime (solid) and heated; (2) the liquid from the above mixture was removed using high pressure, and the liquid stream saved for further product recovery (The liquid stream can be treated with carbonation to capture the calcium as calcium carbonate or the leftover liquid can be used as a source for the chemical 4-ethylphenol); (3) the pH of the solids was adjusted using acid to a pH appropriate for cellulase hydrolysis; and (4) finally, cellulase was added to hydrolyze the cellulose to simple sugars. This method does not have a particle reduction step, as long as the starting material is less than or equal to about 10 inches in length, e.g., bagasse from the mill. The pressing step removes both lignin and the alkali which prevents inhibition of the enzymes used in hydrolysis. In a pilot experiment, only 0.2 g lime/g of dry solid bagasse was used. The method described above was capable of being enzyme solubilized and fermented at a biomass solids loading of 10-30% (w/v). Advantages of this new process include no grinding of bagasse, the low costs of materials, no post-treatment sterilization, accommodation of high loading, easily adaptable to existing sugar industry machinery, and relatively short processing time (less than about 48 hr).

### BRIEF DESCRIPTION OF DRAWINGS

FIG. 1 illustrates the cellulose hydrolysis over time as a percent of the theoretical cellulose hydrolysis using a 1% (w/v) glucan loading for AVICEL® (a synthetic biopolymer) and for lime-treated bagasse.

FIG. 2 illustrates the cellulose hydrolysis over time as a percent of the theoretical cellulose hydrolysis using a 10% (w/v) solids (which equates to a 4% (w/v) glucan) loading for AVICEL® (a synthetic biopolymer), for lime-treated bagasse and for bagasse without lime-treatment (Control).

FIG. 3 illustrates the effect of various levels of glucan loading with AVICEL® (1, 4, 8, and 12% w/v) and with the equivalent amount of bagasse (2.5, 10, 20 and 30% w/v) on the yield of fermentable sugar measured as the percent of the theoretical cellulose hydrolysis.

FIG. 4 illustrates the hydrolysis and fermentation profile for the concentration of glucose, xylose and ethanol in a pilot scale test using 17.6% (w/w) dry solid loading of bagasse after lime pretreatment.

FIG. 5 illustrates the effect of various concentrations of lime (0, 0.02, 0.05, 0.1 and 0.2 g lime/g dry solid bagasse) used for 10% solid loading (or 4% glucan) on the yield of fermentable sugar measured as the percent of the theoretical cellulose hydrolysis at two different time frames, 24 hr and 90 hr.

### MODES FOR CARRYING OUT THE INVENTION

In one embodiment of this method, calcium hydroxide and water were mixed with bagasse and then subjected to high temperature for an appropriate time (30 min-3 hr). Without washing the bagasse after the treatment with lime, the liquid was subsequently removed by squeezing with a high pressure device (such as a sugar mill) with pressures from about 500 psi (pounds per square inch) to about 2000 psi. The liquid contained mainly solubilized lignin components and lime. Ideally, the liquid is removed just after the heating treatment, so that most of the lime was recovered in the liquid portion. The fibrous solid material that remained was then used for hydrolysis by cellulase enzymes. Using this lime pretreatment and pressing step, the structure of the lignocellulosic material was modified such that it was rapidly solubilized by cellulase, even at high solids loading (10-30%) without an inhibitory effect on the cellulase activity. This process is unique among proposed pretreatments for biomass, including other proposed lime treatments, in the ability to remove the lime residue and the solubilization of lignocellulose at greater than 5% solids loading. In addition, this process did not produce enzyme inhibitors. Without wishing to be bound by this theory, it is believed that the pressing step increases the lignin in solution and increases the removal of alkali so that less inhibitors are present for enzyme hydrolysis. This treatment offers numerous advantages over what is currently proposed for conversion of lignocellulosic materials to ethanol, especially by allowing hydrolysis at high solids loading, which is a major advantage for all lignocellulosic, enzymatic conversion, bioethanol processes.

This method should work on all lignocellulosic material, including switchgrass, waste paper, corn grain, corn cobs, corn husks, corn stover, wheat, wheat straw, hay, barley, barley straw, rice straw, sugar cane bagasse, other grasses, sorghum, soy components, trees, branches roots, leaves, wood chips, sawdust, shrubs, bush, and combinations thereof. The starting lignocellulosic material should be of a size less than or equal to about 25 cm length, more preferably less than about 15 cm in length, and most preferably less than about 10 cm in length. Sugarcase bagasse can be used as is. Other materials may have to be chopped to meet this size limitation. However, this method does not require the grinding of any sample into particle sizes less than a centimeter.

Other alkali material could be used for the pretreatment as long as the pH is increased above 11.5 to remove the lignin. Examples of alkali useful for the disclosed method include any mineral alkali, any alkali metal hydroxide, alkaline earth metal hydroxide, or alkaline earth metal oxide, including sodium hydroxide, potassium hydroxide, calcium oxide, calcium hydroxide, lithium hydroxide, rubidium hydroxide, etc. The preferred alkali for bioethanol production is the most economical one, which currently is lime (calcium oxide).

An effective use of lime has many benefits: (1) Alkaline pretreatments, like lime, degrade lignin and leave the cellulose and hemicellulose intact; (2) cellulase inhibitors are not formed from the lignin portion as occurs with acid pretreatments; (3) lime is the least expensive base that could be used; (4) lime is more environmentally friendly than other potential bases; (5) lime is relatively easy to recover as calcium salt; and (6) use of lime in industry is known.

The temperature of the alkali pretreatment step depends on the concentration of the alkali and the biomass, and depends on the time for the process. For the current method, the range of temperature is from about 50° C. to about 150° C., with a preferred range of about 80° C. to about 140° C. The time for the alkali pretreatment is from about 20 min to about 10 hr, with the preferred range of about 20 min to 6 hr.

For the saccharification step, the temperature and pH must be adjusted to levels compatible with the enzymes to be added for hydrolysis. The potential range in temperature for enzymatic hydrolysis is from about 20° C. to about 70° C., with a preferred range of about 28° C. to about 55° C. The pH range can be from about 4 to about 7, with a preferred range of about 4.5 to about 5.5. The pH can be adjusted after the alkali pretreatment with an acid, for example, with sulfuric acid, hydrochloric acid, or hydrofluoric acid. The only limitation is whether the acid would form salts that would inhibit the enzymes. For bioethanol production, sulfuric acid is currently preferred as being the most cost-effective.

For fermentation of the sugars produced by the saccharification step, any known fermentation organism can be used, including yeast (Saccharomyces cerevisiae), and other microorganisms (recombinant Escherichia coli, Zymomonas mobilis, Bacillus stearothermophilus, and Pichia stipitis), either naturally-occurring or genetically modified. In addition, to increase the ethanol recovery an inexpensive sugar source may be added to the fermentation step, e.g., molasses.

### Example 1

### Materials and Methods

Lignocellulosic Material. Sugarcane bagasse (bagasse) was collected from a sugarcane bagasse pile at a local sugar mill in Louisiana. The bagasse was used “as-is”, with sizes ranging from about several millimeters to about 10 cm length. All weights were based on dry weights, where the obtained weight was corrected by using a moisture analyzer at 105° C. (Computrac MAX 1000, Arizona Instrument Corporation, Tempe, Arizona) to determine retained moisture.

Treatment with Lime. Bagasse was mixed with hydrated lime powder (Ca(OH)₂; Fisher Scientific, Fair Lawn, N.J.), and then deionized water was added to produce the desired bagasse to water ratio and bagasse to chemical loading ratio. For example, 1 g of dry solid bagasse was mixed with 0.2 g of dry solid lime powder and with 10 g of deionized water to make a 1:10 bagasse to water ratio and a 1:0.2 bagasse to lime loading ratio. This mixture was heated to 121° C. for 1 hr in an autoclave. Immediately after treatment, without washing, the mixture was pressed in a pilot scale sugar milling tendam (Farrel Corp., Ansonia, Conn.) which consisted of 3 horizontal rolling shafts, each 30 cm wide and 15 cm in diameter, to extract liquid. The tendam produced pressures of about 20 and about 41 tons pressure per foot long of bagasse lined on the rolls, or pressures from about 1000 to about 2000 pounds per square inch (psi). It is believed that pressures sufficient to achieve the desired result range from about 500 psi to about 2000 psi, with the preferred range being about 1000 psi to about 2000 psi. The resulting fibrous, de-watered, unwashed, lime pretreated bagasse was stored at 4° C. from a few days to weeks, but less than 2 months, before use for enzyme hydrolysis.

Composition of treated bagasse. Structural carbohydrates and lignin of bagasse before and after treatment were determined as described by the National Renewable Energy Laboratory (NREL at the website, http://www.eere.energy.gov/biomass/analytical_procedures.html, accessed November 2006.)

Enzyme hydrolysis. The lime-treated bagasse was subjected to enzyme hydrolysis, without sterilization in large flasks. The pH was adjusted with sulfuric acid to an enzyme optimum pH 4.8-5.2. The pH change by residual lime discharge from the treated bagasse was monitored with an extra sample and additional sulfuric acid was added if necessary to maintain the pH range close to optimum for enzymatic activity. Enzymatic hydrolysis of the cellulose residue was conducted using commercially available enzymes, Spezyme CP (Genencor International Co., Cedar Rapids, Iowa) and Novo188 (Novozyme; Salem, Va.). The enzyme activity was measured as Filter Paper Units/gram solid (FPU/g solid) according to NREL procedure. The cellobiase activity was given by the manufacturer. Enzyme saccharifications were measured by NREL methods (NREL at the website, http://www.eere.energy.gov/biomass/analytical_procedures.html), accessed November 2006. The concentrations of the enzymes used were cellulase (60 FPU/g of glucan) and cellobiase (64 CBU/g of glucan). The amount of hydrolysis was followed over time. In the event of greater than 10% (w/w) solid loading, the flasks were agitated at 180 rpm during enzyme hydrolysis and at 100 rpm for fermentation because of the high viscosity of the slurry. AVICEL® (Ph-102; FMC Biopolymer, Philadelphia, Pa.) was used as a control. The following formula was used to calculate percent of theoretical of cellulose hydrolysis (NREL LAP-008; http://www.eere.energy.gov/biomass/analytical_procedures.html accessed November 2006.)

$$\% Yield = \frac{( Glucose ) + 1.053 \times ( Cellobiose )}{1.111 \times f \times ( Biomass )} \times 100$$

In the above formula, “glucose” represents the residual glucose concentration (g/L); “Cellobiose” represents the residual cellobiose concentration (g/L); “Biomass” represents dry Biomass (in most experiments, bagasse) concentration at the beginning of the saccharification (g/L); “f” is the cellulose fraction in dry bagasse (g/g) as calculated from the composition analysis; and “1.053” is the multiplication faction that convert cellobiose to equivalent glucose.

Fermentation. Glucose released from the enzymatic hydrolysis was fermented to ethanol using commercially available yeast, Saccharomyces cerevisiae, a Fleischmann's product (Distributor; ACH Food Companies, Inc. Memphis, Tenn.). Yeast cells were loaded at 10⁷ CFU/ml. Temperature was maintained at 30° C. during fermentation. The theoretical yield was calculated using the following formula.

$$\% Yield = \frac{{( EtOH )}_{f} - {( EtOH )}_{0}}{0.51 ( f \times ( Biomass ) \times 1.111 )} \times 100$$

In the formula, “(EtOH)_{f}” represents the ethanol concentration (g/L) at the end of the fermentation minus any ethanol produced from the enzyme and medium; “(EtOH)₀” represents the ethanol concentration (g/L) at the beginning of the fermentation which should be zero; “(Biomass)” represents the dry biomass (in most experiments, bagasse) concentration (g/L) at the beginning of the fermentation; “f” represents the cellulose fraction in dry biomass (g/g) as calculated from the composition analysis; “0.51” is the conversion factor for glucose to ethanol based on stoichiometric biochemistry of yeast; and “1.111” is the conversion factor for cellulose to equivalent glucose.

Sugar and Ethanol Analysis. Samples were obtained at several time intervals during hydrolysis and fermentation. Ethanol, xylose, glucose, arabinose and cellobiose were determined by the use of a Waters system HPLC with an Aminex-HPX-87K Bio-Rad column (Bio-Rad Lab., Hercules, Calif.) run at 85° C. with K₂HPO₄ as eluent, at a constant flow rate of 0.6 ml/min. The Refractive Index was used for detection of sugars. The concentration of sugars and ethanol from the HPLC was used to calculate cellulose hydrolysis and fermentation yield.

Pilot Bioethanol Production. Pilot-scale bioethanol production was demonstrated using a 120 L sugar crystallizer as a reactor vessel, equipped with the ability to mix and to control the temperature. The reactor had a horizontal rotary shaft mounted with paddles for mixing. The mixing speed was set at 8 rpm per minute. To achieve an optimum pH for the enzymes, sulfuric acid was added. Tap water was added to get 18% (w/w) dry solid loading. The initial enzyme loading was 60 FPU/g glucan (Spezyme CP) and 64 CBU/g glucan (Novo188), and the temperature raised to 50° C. During the initial run, a pH control problem was encountered in the early stages of enzyme hydrolysis that caused the enzymes to become inactivated. The problem was confirmed by a separate enzyme activity test of the collected samples. (data not shown). Additional enzymes (30 FPU/g glucan Spezyme CP and 32 CBU/g glucan Novo188) were added at 19 hr. For fermentation, the temperature was lowered to about 30° C., and at about 42 hr the yeast organisms were added. The temperature was maintained at about 30° C. until fermentation was completed. Samples were collected at desired intervals and stored frozen for HPLC analysis. After fermentation, the mixture was filtered, and the filtrate was used for alcohol recovery. Alcohol was recovered using a sugar mill style evaporator (pilot scale) for the initial distillation, and then further purified in a laboratory scale distillation apparatus.

### Example 2

### Composition of Bagasse Before and After Lime Treatment

The lime-pretreated fibrous material after milling (or pressing) contained 60 to 70% dry solids. The composition of the bagasse before and after the lime treatment and pressing was determined as described above by the NREL procedure. The results are shown in Table 1.

TABLE 1
Composition analysis of bagasse before and
after lime treatment (% dry wt)

| Group | Components | Raw bagasse | Pretreated |
|---|---|---|---|
| Cellulose | Glucan | 30.3 | 34.1 |
| Hemi- | Xylan | 19.2 | 17.9 |
| Cellulose | Arabinan | 1.2 | 1.3 |
| | Mannan | 0.5 | 0.5 |
| Lignin | Acid-insoluble lignin | 24.5 | 16.6 |
| | Acid soluble lignin | 5.0 | 4.7 |
| | Total lignin | 29.5 | 21.3 |

| | | |
|---|---|---|
| Ash | 2.5 | 8.7 |

| | | |
|---|---|---|
| Total | 87.4 | 90.2 |

The treatment with lime and pressing apparently did not remove either the cellulose or hemicelluloses, only a large percent of the lignin. About 93% (w/w) of the xylan was retained, but the other minor sugars did not change concentration. However, the process removed 28% of the lignin. In one experiment, an increase of ash content was observed possibly due to calcium trapped in the cellulose matrix as the treated bagasse was only squeezed under pressure but not washed. The lignin removal was almost twice that reported by Chang et al. (1998).

### Example 3

### Cellulose Enzyme Hydrolysis to Glucose at 2.5% Solid Loading

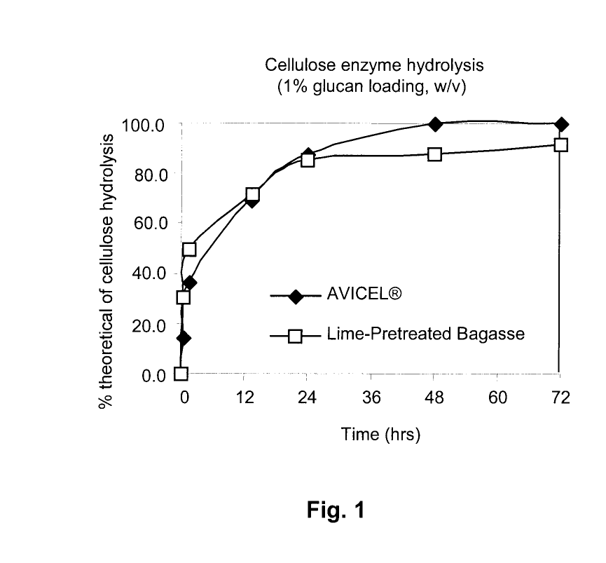

Although most studies use cellulase loadings for hydrolysis of less than 40 FPU/g of glucan, because sugarcane bagasse is more recalcitrant than corn stover, a higher dose of enzymes was used in this experiment. Chang et al. (1998) reported that sulfuric acid was not effective for pH adjustment and enzyme hydrolysis in their lime treatment of biomass, and used glacial acetic acid to lower the pH. However, they reported an inhibitory effect on the cellulases due to calcium acetate that was formed as salt concentration increased. Another group reported that the calcium acetate from pH adjustment with glacial acetic acid did not affect the enzyme activity against corn stover (Karr, William E., and Holtzapple, Mark T., 2000. Using lime pretreatment to facilitate the enzyme hydrolysis of corn stover. Biomass and Bioenergy, 18: 189-199). However, in our experiment, sulfuric acid was found to be an effective pH reducing agent and did not result in inhibition of enzyme hydrolysis. FIG. 1 shows the cellulose hydrolysis over time as a percent of theoretical cellulose hydrolysis using a 1% glucan (a generic name for a glucose polymer which in this experiment is related to the amount of cellulose in the bagasse; and 1% glucan is equivalent to 2.5% solid loading containing 40% glucan) loading for AVICEL® (as a control) and a 2.5% loading for lime-treated, pressed bagasse as described above. After 6 hr, cellulase had released about 60% of glucan as glucose from the lime-treated bagasse, and the conversion reached a plateau at 24 hr at 84% cellulose hydrolysis. When compared with AVICEL®, the course of enzyme hydrolysis was similar, indicating no inhibition of hydrolysis by the pretreatment of the bagasse.

### Example 4

### Cellulose Enzyme Hydrolysis to Glucose at 10% Solid Loading

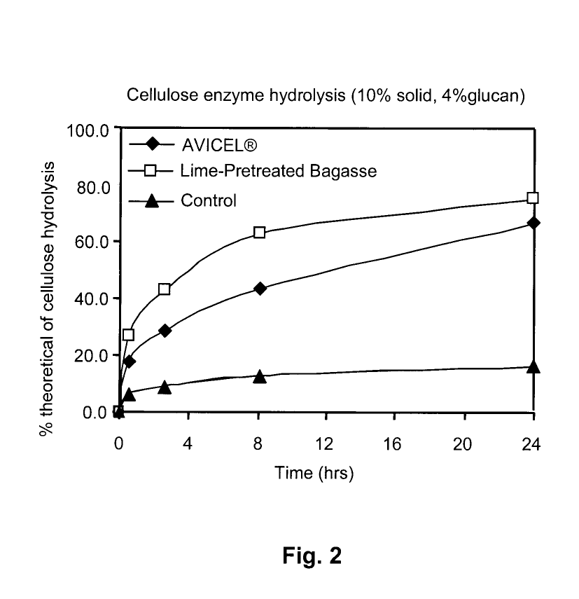

FIG. 2 illustrates the percent theoretical of cellulose hydrolysis using 10% solids, or a 4% glucan loading (w/v). As shown in FIG. 2, at a level of 4% glucan (equivalent to 10.0% solid loading containing 40% glucan), the treated bagasse hydrolyzed better than AVICEL®. The bagasse treated under the same conditions, without the lime pretreatment, showed only a 16% cellulose hydrolysis (FIG. 2, Control).

### Example 5

### Ethanol Production from Pretreated Bagasse

An experiment was conducted to see how much ethanol could be made from bagasse, pretreated with the above process, then incubated for 16 hr with enzyme to make glucose (the saccharification step), and then subsequently incubated with yeast cells to ferment the glucose to ethanol for up to 44 hr (fermentation). To determine the maximum amount of ethanol that could be produced from the treated bagasse, a series of hydrolyses with increasing solids loading, 10% to 30% (w/w) were conducted. Only with 10% (w/w) dry solids was any free liquid visible; i.e., at 20% (w/w) or higher no free liquid was observed at the start of the hydrolysis. Enzyme hydrolysis was started at 50° C. (for 16 hr), prior to yeast addition to produce liquid for the fermentation and to discourage microbial contamination by mesophilic bacteria in the initial stage. Fermentation was allowed to proceed for 28 hr post-inoculation. After addition of the fermentation organisms, the 10% (w/w) dry solid samples liquefied within 1 hr. However, the 25% (w/w) solid loading samples liquefied in less than 12 hr, and for 30% solids loading a slurry liquid enough for pumping was obtained after 12 hr. With 25% solid loading, 3.4% ethanol (w/v, 4.3% by volume) was produced. For economical distillation at least 4% ethanol (w/w, or 5% (v/v)) in the fermentation beer is desired. (Katzen, R., Madson, P. W., Moon, G. D. 1999. Alcohol distillation—The fundamentals. In: Jacques, K. A., Lyons T. P., Kelsall, D. R., editors. The Alcohol Textbook. Nottingham: Nottingham University Press, pp 103-125; and Wingren, A., Galbe, M. Zacchi, G. 2003, Techno-economic evaluation of producing ethanol from softwood: comparison of SSE and SHF and identification of bottlenecks. Biotechnol. Prog. 19:1109-1117). The ethanol concentration from 25% loading was low for economical distillation, but was ethanol produced only from the cellulose. For economical distillation, a fed-batch approach may be necessary to achieve higher concentrations of ethanol when only glucose from cellulose is fermented. There are other ways are to reach the higher ethanol yield, such as using the sugars derived from hemicellulose or supplementing with a small amount of cheap sugar sources, e.g., cane backstrap molasses.

As shown in Table 2, for up to 12% glucan, which is equivalent to 30.0% solid loading containing 40% glucan, the treated bagasse was subjected to enzyme hydrolysis with Spezyme CP (60 FPU/g of glucan) and Novo188 (64 CBU/g of glucan). This table confirms that glucose from the cellulose in the lime-pretreated bagasse as described above was easily fermented to ethanol.

TABLE 2
Enzyme hydrolysis and yeast fermentation with high solid loading

| | % Solid loading (w/v) | | | | | | | |
|---|---|---|---|---|---|---|---|---|
| | 10 | | 20 | | 25 | | 30 | |
| | Glucose | | Glucose | | Glucose | | Glucose | |
| Time | (%, | Ethanol | (%, | Ethanol | (%, | Ethanol | (%, | Ethanol |
| (hr) | w/v) | (%, w/v) | w/v) | (%, w/v) | w/v) | (%, w/v) | w/v) | (%, w/v) |
| 0 | 0 | | 0 | | 0 | | 0 | |
| 16 | 2.4 | | | | 4.8 | | 3.3 | |
| 24 | | 1.4 | | 2.6 | | 2.9 | | 2.2 |
| 44 | 0 | 1.6 | 0 | 3.0 | 0 | 3.4 | | 3.3 |
| | | (70% of | | (65% of | | (60% of | | (49% of |
| | | theoretical | | theoretical | | theoretical | | theoretical |
| | | yield from | | yield from | | yield from | | yield from |
| | | cellulose) | | cellulose) | | cellulose) | | cellulose) |

### Example 6

### Effect of Solids Concentration on Ethanol Production

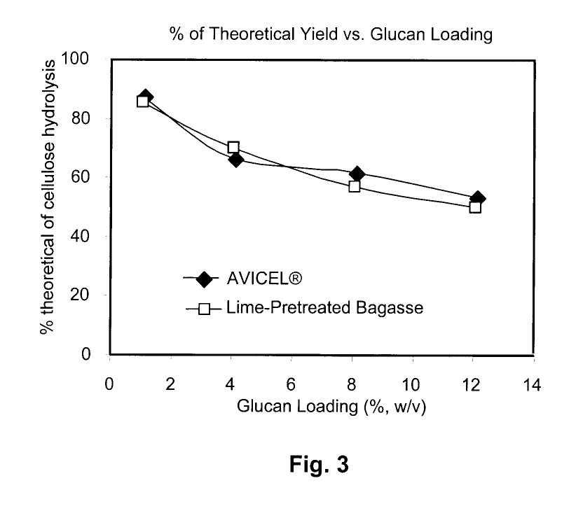

An experiment was conducted to analyze the effect of solids concentration on the yield of fermentable sugar using the pretreated process of bagasse described above. Four concentrations of glucan loading with AVICEL® (1, 4, 8, 12%) and equivalent of lime-treated, pressed sugar cane bagasse (2.5, 10, 20, and 30%) were hydrolyzed in order to analyze the effect of solids loading on the yield of fermentable sugar. FIG. 3 shows the results. A fixed percentage of the cellulose remained unavailable to enzyme hydrolysis in both the treated bagasse and the AVICEL® samples. More importantly, our treatment method did not produce fermentation inhibitors since the fermentation was the same as with the pure cellulose, AVICEL®. End products such as glucose and cellobiose are known to be inhibitors of enzyme hydrolysis in high solid loading. Although yeast was added to relieve the end product inhibition, the % of theoretical ethanol yields from cellulose still decreased with an increase in solid loadings. This decrease in conversion may be related to other factors, such as differences in diffusion rate, water availability, osmotic pressure and/or sugars from hemicellulose in high solid loading.

### Example 7

### Pilot Scale Bioethanol Production

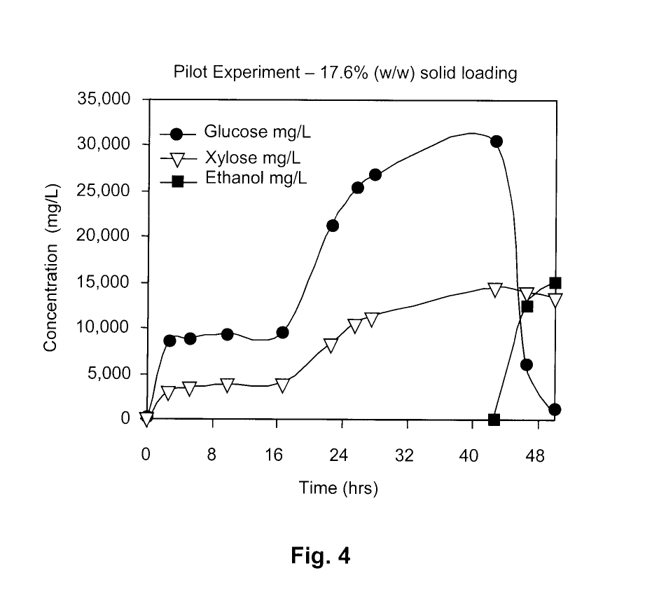

A pilot scale test of cellulosic ethanol (72 L with 17.6% (w/w) dry solid loading) was conducted using S. cerevisiae as the organism of choice. The process used 17.6% solids (containing 34.05 g glucan/100 g dry solids) loading at a 72 L scale. The initial enzyme loading was 60 FPU/g glucan (Spezyme CP) and 64 CBU/g glucan (Novo188). However, a pH control problem (described below) was encountered in the early stages of enzyme hydrolysis that caused the initial enzymes to become inactivated. Because a pH control system was not installed to the reactor, the pH of the solid increased from 5.0 at 0 hr to 7.3 because of discharge of lime from the bagasse at 2 hr. Once discovered, additional sulfuric acid was sprayed on the biomass to lower the pH again back to 5. The enzymes were thus inactivated from hour 2 until more enzymes were added at hour 16. Additional enzymes (30 FPU/g glucan Spezyme CP and 32 CBU/g glucan Novo188) were added at 19.3 hr. Yeast organisms were added at 42.5 hr. FIG. 4 shows the hydrolysis/fermentation profile during the process. As shown in FIG. 4, after additional enzyme was added, the glucose concentration rapidly increased and the saccharification component was complete in about 42 hr. The enzyme conversion of cellulose and hemicellulose was 49.3 and 38.6% of theoretical yields at 42 hr, respectively. The % of theoretical yield of cellulose conversion to ethanol was 44.8%. Fermentation was complete in 8 hr after addition of yeast as shown in FIG. 4. After fermentation, the liquid (beer) was pre-filtered with an 8 mesh screen (2.34 mm) and the filtrate was used for alcohol recovery. Alcohol was recovered using a sugar mill style evaporator for the initial purification, and then further purified using a lab scale distillation. About 1 L of 70% (v/v) cellulosic ethanol was recovered.

As shown in FIG. 4, the saccharification component was complete in about 42 hr (the addition of enzyme to the treated bagasse to make glucose from glucan). Yeast organisms were then added, and fermentation was complete in 8 hr as shown in Table 3. The yield of cellulose conversion to ethanol was 44.8% of the theoretical yield, calculated as shown above. The cellulose conversion of 50% of theoretical in the table is somewhat lower than the 60% conversion that was predicted from the earlier laboratory scale experiments as shown in Table 2 above.

TABLE 3
Results from SHF of pilot scale trial on bagasse.

| | % of Theoretical Yield | | |
|---|---|---|---|
| Time | | | Ethanol |
| (hr) | Glucan | Xylan | from Glucan |
| 26 | 43.6 | 27.8 | — |
| 42 | 49.3 | 38.6 | — |
| 50 | — | — | 44.8 |

### Example 7

### Effect on Cellulose Hydrolysis of Various Concentrations of Lime During Pretreatment

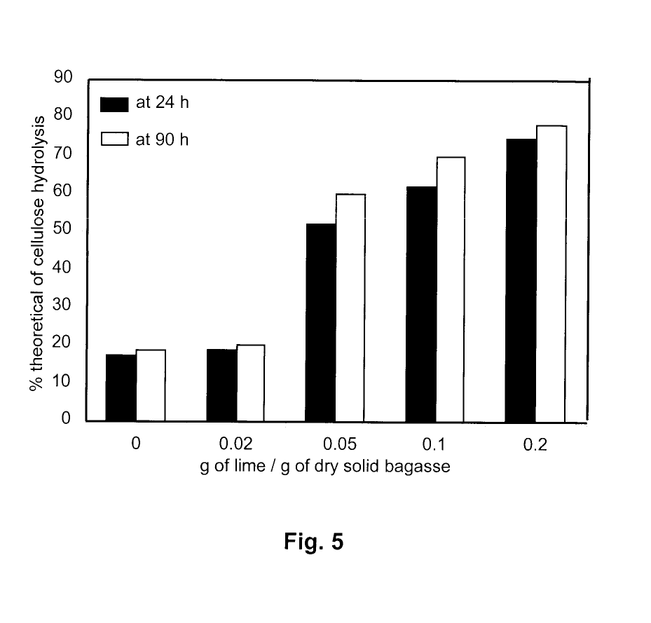

An experiment was conducted to test various concentrations of lime (0, 0.02, 0.05, 0.1 and 0.2 g lime/g dry solid bagasse) when using 10% solid loading of bagasse. As shown in FIG. 5, at 4% glucan, which is equivalent to 10.0% solid loading containing 40% glucan, increased lime addition enhanced the enzyme hydrolysis of cellulose. Although the literature had reported an optimal lime to bagass ratio of 0.1 g lime/g dry solid bagasse (at 40 mesh screened), this experiment indicated that 0.2 g lime/g bagasse (as is) is preferred.

The complete disclosures of all references cited in this specification are hereby incorporated by reference. In the event of an otherwise irreconcilable conflict, however, the present specification shall control.

We claim:

1. A method for producing fermentable sugars from lignocellulosic biomass, said method comprising the sequential steps of:
(a) Treating the biomass with an aqueous alkali solution at ambient pressure and at greater than ambient temperature for a time sufficient to enhance the susceptibility of the biomass to a subsequent saccharification enzyme hydrolysis step;
(b) Pressing the treated mixture at a pressure great enough to remove sufficient water, alkali, and lignin from the biomass to enhance the susceptibility of the de-watered biomass to a subsequent saccharification enzyme hydrolysis step; and
(c) Contacting the de-watered biomass with one or more saccharification enzymes under conditions conducive to producing fermentable sugars.

2. The method of claim 1, wherein the biomass is selected from the group consisting of switchgrass, waste paper, corn grain, corn cobs, corn husks, corn stover, wheat, wheat straw, hay, barley, barley straw, rice straw, sugar cane bagasse, other grasses, sorghum, soy components, trees, branches roots, leaves, wood chips, sawdust, shrubs, bush, and combinations thereof.

3. The method of claim 1, wherein the biomass is of a size less than about 25 cm, more preferably less than about 15 cm, and most preferably less than about 10 cm.

4. The method of claim 1, wherein the biomass is of a size less than about 10 cm.

5. The method of claim 1, wherein the biomass is sugarcane bagasse.

6. The method of claim 1, wherein the biomass is unground.

7. The method of claim 1, wherein the alkali solution is an aqueous solution of an alkali selected from the group consisting of sodium hydroxide, potassium hydroxide, calcium oxide, calcium hydroxide, lithium hydroxide, and rubidium hydroxide.

8. The method of claim 1, wherein the alkali is calcium oxide.

9. The method of claim 8, wherein the ratio of alkali to biomass is between about 0.05 and about 0.2 grams alkali to about 1.0 gram dry solid biomass.

10. The method of claim 8, wherein the ratio of alkali to biomass is about 0.2 grams alkali to about 1.0 gram dry solid biomass.

11. The method of claim 1, wherein the mixture of step (a) is conducted at a temperature of between about 50° C. and about 150° C.

12. The method of claim 1, wherein the mixture of step (a) is conducted at a temperature of between about 80° C. and about 140° C.

13. The method of claim 1, wherein step (a) is conducted for a time between about 20 minutes and about 10 hours.

14. The method of claim 1, wherein step (a) is conducted for a time between about 20 minutes and about 6 hours.

15. The method of claim 1, wherein step (a) is conducted at a temperature between about 80° C. and about 140° C., and for a time between about 20 minutes and about 6 hours.

16. The method of claim 1, wherein the pressing step (b) is conducted at a pressure between about 500 psi and about 2000 psi.

17. The method of claim 1, wherein the pressing step (b) is conducted at a pressure between about 1000 psi and about 2000 psi

18. The method of claim 1, wherein step (c) comprises: lowering the pH of the pressed material with a mineral acid, and lowering the temperature of the pressed material, such that the pH and the temperature are compatible with saccharification enzyme hydrolysis.

19. The method of claim 18, wherein the acid is selected from the group consisting of sulfuric acid, hydrochloric acid and hydrofluoric acid.

20. The method of claim 18, wherein the acid is sulfuric acid.

21. The method of claim 18, wherein the pH is lowered to between about 4 and about 7.

22. The method of claim 18, wherein the pH is lowered to between about 4.5 and about 5.5.

23. The method of claim 18, wherein the temperature is lowered to between about 20° C. and about 70° C.

24. The method of claim 18, wherein the temperature is lowered to between about 28° C. and about 55° C.

25. The method of claim 1, wherein one or more saccharification enzymes comprise one or more cellulases.

26. The method of claim 1, wherein the biomass is between about 10% and about 30% solids of weight of biomass per volume.

27. A method for producing ethanol, comprising the steps of:
(a) Producing fermentable sugars from lignocellulosic biomass by the method of claim 1; and
(b) Contacting the fermentable sugars under suitable fermentation conditions with a suitable fermentation organism to produce ethanol.

28. The method of claim 27, additionally comprising the step of adding an additional sugar source to the fermentable sugars.

29. The method of claim 28, wherein the additional sugar source comprises molasses.

30. The method of claim 27, wherein the fermentation organism is a wild-type or modified organism selected from the group consisting of Saccharomyces cerevisiae, Escherichia coli, Zymomonas mobilis, Bacillus stearothermophilus, and Pichia stipitis.

31. The method of claim 27, wherein the fermentation organism is Saccharomyces cerevisiae.
